(19) United States
(12) Patent Application Publication
(10) Pub. No.: US 2025/0320565 A1
(43) Pub. Date: Oct. 16, 2025

(54) DETECTING ANTIBIOTIC AND/OR CHEMICAL USE IN AQUATIC ANIMALS USING EPIGENETIC MEANS

(71) Applicants: Evonik Operations GmbH, Essen (DE); Deutsches Krebsforschungszentrum Stiftung des Öffentlichen Rechts, Heidelberg (DE)

(72) Inventors: Annika LINGENBERG, Heidelberg (DE); Sina LYKO-TÖNGES, Heidelberg (DE); Frank LYKO, Heidelberg (DE); Rose WHELAN, Ambrosden Oxfordshire (GB); Florian BÖHL, Neckargemünd (DE); Suki ROY, Singapore (SG); Sanjanaa NAGARAJAN, Singapore (SG); Christos GIATSIS, Aschaffenburg (DE); Stefan PELZER, Gütersloh (DE)

(73) Assignees: Evonik Operations GmbH, Essen (DE); Deutsches Krebsforschungszentrum Stiftung des Öffentlichen Rechts, Heidelberg (DE)

(21) Appl. No.: 18/881,607

(22) PCT Filed: Jul. 4, 2023
(86) PCT No.: PCT/EP2023/068428

§ 371 Date: Jan. 6, 2025

(30) Foreign Application Priority Data
Jul. 8, 2022 (EP) 22183743.8

(51) Int. Cl. C12Q 1/6888; C12Q 1/6809

(52) U.S. Cl. CPC C12Q 1/6888; C12Q 1/6809; C12Q 2600/142

(57) ABSTRACT

A method of determining if an aquatic test animal and/or test animal from which a product is derived has been treated and/or is currently undergoing treatment with at least one antibiotic and/or veterinary chemical includes: (a) determining a test methylation profile from genomic material contained in a biological sample obtained from the test animal and/or animal-derived product; and (b) comparing the test methylation profile obtained from (a) with a reference methylation profile obtained from a control animal of the same biological taxon of the test animal, where the control animal was not treated and/or is not currently undergoing treatment with at least one antibiotic and/or veterinary chemical, wherein a difference in the test methylation profile of (a) compared to the reference methylation profile from the control animal is indicative of the test animal having been treated and/or is currently undergoing treatment with at least one antibiotic and/or veterinary chemical.

### FIELD OF THE INVENTION

The present invention relates to a method for detecting the use and/or administration of at least one antibiotic and/or chemical to an aquatic animal using epigenetic means. In particular, the method is capable of detecting the use of at least one antibiotic or the administration of at least one antibiotic and/or chemical during the breeding of at least one aquatic animal by determining the methylation status of a CpG site in a test animal and comparing the resultant methylation status with a reference methylation status of a control animal which was bred without the use of or administration of an antibiotic and/or chemical.

### BACKGROUND OF THE INVENTION

Antibiotics are used for the treatment of bacterial diseases in human and veterinary medicine. Antibiotics have also been used prophylactically at low levels in feed or water to improve growth rates and mortality levels in livestock. However, overuse of antibiotics especially in agriculture, has been associated with an increase in antibiotic resistant bacteria, which will eventually impair the ability to treat bacterial diseases in humans and animals alike. Additionally, antibiotic usage has been associated with other unintended and undesirable physiological consequences like low birth weight in infants from antibiotic treated mothers, increased disease susceptibility, and the like. Tools for assessing not only current, but historical antibiotic use would therefore be of high interest in livestock production for the purpose of guiding breeding, feeding, veterinary and/or management practices. Additionally, analysis of meat and meat products to assess historic antibiotic use in the individual animal prior to slaughter could be of high interest for auditing, certifying and labelling of meat products.

To date antibiotic testing involves chemical assays to find residues of antibiotics present in the blood or tissues of animals. However, most antibiotics used for growth promotion are either added to the feed or water and most of these classes are poorly absorbed in the gastrointestinal system, so residue testing in blood or tissues may not capture usage of all antibiotic classes. There is also currently no method by which aquatic animals or meat products can be tested for previous antibiotic usage unless antibiotic residues remain in the residues remain in the animal (ie. blood or tissues) or the aquatic environment (ie. pond or tank).

Accordingly, there is a need in the art for a simple and accurate means of detection of current and past antibiotic and/or drug use in animals, particularly aquatic farming animals.

Epigenetics is the study of inherited traits caused by mechanisms other than changes in the underlying DNA sequence. In other words, epigenetic marks “orchestrate” our genes. Epigenetic marks can be either chemical (e.g. methylation), protein-based (e.g. histones) or a combination of the two. During development and cell differentiation, DNA methylation is dynamic, but some DNA methylation patterns may be retained as a form of epigenetic memory, accumulated and/or inherited to next generation. Those changes might be responsible for heritable changes in gene activity as DNA methylation events have been shown to be regulation mechanisms associated with gene silencing, expression, chromatin remodeling or imprinting. Epigenetics is attractive for animal breeding as it may identify causality and heritability of complex traits and diseases. DNA methylation patterns are modified along the life of an individual by environmental forces like diet, stress, drugs, or pollution among many others. Some environments are more likely to increase certain methylation patterns, and these patterns could contribute to the epigenetic and/or phenotypic variation between individuals.

Several studies have shown that antibiotic exposure can change epigenetic markers in eukaryotic cells. For example, in a study examining the relationship between antibiotic usage during pregnancy and birth weight outcomes, it was found that there were specific regions of the genome that were differentially methylated in response to antibiotic exposure (Vidal et al., 2013). A study conducted with cultured plant cells observed changes in global DNA methylation when the cells were exposed to the antibiotic kanamycin (Bardini et al., 2003). However, there is no mention in the art of epigenetic changes that may occur due to current and/or past antibiotic and/or drug use in animals, particularly aquatic animals.

Accordingly, there is still a need in the art for a simple and accurate method for detection of current and/or past antibiotic and/or drug use in animals, particularly aquatic animals.

### DESCRIPTION OF THE INVENTION

The present invention solves the problems above by providing a means of using alterations in DNA methylation patterns to develop novel analytics for assessment of antibiotic or other chemical usage in aquatic agricultural animal production, meat and/or meat products. In particular, the means comprises a method of detecting the use of antibiotics and/or veterinary chemicals in a test animal and/or a test animal from which a product is derived by comparing the methylation status of at least one CpG site in the test animal and the corresponding CpG site in a control animal and/or a control animal from which a product is derived, where no antibiotics and/or veterinary chemicals were used on the control animal and, wherein the presence of hypomethylation or hypermethylation at the CpG site in the test animal is indicative of the test animal having been treated with antibiotics and/or veterinary chemicals. The method according to any aspect of the present invention is a simple and accurate method for detection of current and/or past antibiotic and/or drug use in animals, specifically aquatic animals.

The present invention is based on the finding that contact with antibiotics and/or veterinary chemicals to an animal, results in stress in the internal environment of the animal and this can permanently change the genome of the animal through epigenetics. In particular, the capability to adapt to the environment (i.e. presence of antibiotics and/or veterinary chemicals) and maintain the adapted biological pattern depends on epigenetic mechanisms, including DNA methylation. In particular, the present invention is based on the finding that contact with antibiotics and/or veterinary chemicals may also result in changes in epigenetic mechanisms of the animal, including DNA methylation patterns and these patterns may also be passed down to the different products that may derive from the animal.

The inventors have unexpectedly found that this property can be utilized to identify “epigenetic fingerprints” on the genome that are specific to the presence of antibiotics and/or veterinary chemicals of not just one animal but possibly all the animals that are brought into contact with at least one of antibiotic and/or veterinary chemical. For example, these ‘epigenetic fingerprints’ may be specific not only for the animal that was brought into contact with antibiotics and/or veterinary chemicals but also for any product that stems from the very animal (an individual). Based on these findings, the present invention provides means to detect use of antibiotics and/or veterinary chemicals in animals, in particular aquatic animals, for example rearing aquatic animals from which an animal-derived product comes from. In this way, the method according to any aspect of the present invention may then be used to accurately and reliably determine if any test animal or product therefrom has been brought into contact or treated with at least one antibiotic and/or veterinary chemical. The method according to any aspect of the present invention may then use DNA methylation analysis to also differentiate usage of antibiotics prophylactically (growth promotion) from usage of antibiotics therapeutically. DNA methylation analysis may also be a step, in elucidating, whether an animal treated with veterinary chemicals and/or antibiotics was given a withdrawal period from the veterinary chemical and/or antibiotic prior to killing the animal or collecting the animal for use or consumption or differentiate usage of different classes of antibiotics. The method according to the present invention may also be used for assessment of current and historic antibiotic usage in aquatic livestock animals or meat to investigate epigenetic markers which can then subsequently be used for the creation of analytics to test antibiotic usage in aquatic livestock species. Further, the method according to any aspect of the present invention may then be used to predict and avoid undesired traits contributed by use of antibiotics and/or veterinary chemicals to the animal welfare and to a more sustainable aquatic livestock production. The method according to any aspect of the present invention may also be used to differentiate the route of administration of the antibiotic to the animal.

According to one aspect of the present invention, there is provided a method of determining if a test animal and/or a test animal from which a product is derived has been treated and/or is currently undergoing treatment with at least one antibiotic and/or veterinary chemical, the method comprising:
(a) determining a test methylation profile from the genomic material contained in a biological sample obtained from the test animal and/or the animal-derived product; and
(b) comparing the test methylation profile obtained from (a) with a reference methylation profile obtained from a control animal, where the control animal was not treated and/or is not currently undergoing treatment with at least one antibiotic and/or veterinary chemical,
   wherein a difference in the test methylation profile of (a) compared to the reference methylation profile from the control animal, is indicative of the test animal having been treated and/or is currently undergoing treatment with at least one antibiotic and/or veterinary chemical; and
   wherein the test animal is an aquatic animal and the veterinary chemical is an anti-parasitic, an anti-viral, a feed additive, a water additive, a disinfectant, glutaraldehyde, and/or formalin used in aquaculture.

In particular, the method according to any aspect of the present invention can be used to detect antibiotics-related methylation differences for any stage/age of an aquatic animal, particularly aquatic livestock. More in particular, the method according to any aspect of the present invention can trace the use of antibiotics and or veterinary chemicals used commonly in aquaculture in an aquatic animal, particularly aquatic livestock. According to a further aspect of the present invention, there is provided a method of determining if a test animal and/or a test animal from which a product is derived has been treated and/or is currently undergoing treatment with at least one antibiotic and/or veterinary chemical, the method comprising:
(a) determining the methylation status of at least one CpG site in genomic material contained in a biological sample obtained from the test animal,
(b) comparing the methylation status of the CpG site from (a) with that of a control animal which was not treated and/or is not currently undergoing treatment with at least one antibiotic and/or veterinary chemical,
   wherein a difference in the test methylation status of (a) in the test animal compared to the CpG site in the control animal is indicative of the test animal having been treated and/or is currently undergoing treatment with at least one antibiotic and/or veterinary chemical; and
   wherein the test animal is an aquatic animal and the veterinary chemical is an anti-parasitic, an anti-viral, a feed additive, a water additive, a disinfectant, glutaraldehyde, and/or formalin used in aquaculture.

The difference in methylation status according to any aspect of the present invention is hypomethylation or hypermethylation of the CpG site in the test animal and the hypomethylation or hypermethylation of the CpG site is indicative of the test animal having been treated and/or is currently undergoing treatment with at least one antibiotic and/or veterinary chemical.

As used herein, the term “aquatic animal” refers to any organism that lives entirely in water or that lives predominantly in water, especially compared with terrestrial animals. In particular, the aquatic animal according to any aspect of the present invention may be any animal in the animal kingdom that lives predominantly in water. These aquatic animals may live in different water forms, such as seas, oceans, rivers, lakes, ponds, etc. More in particular, the aquatic animal according to any aspect of the present invention may be any aquatic livestock and may be any fish, aquatic molluscs, or aquatic crustaceans, at all life stages, including eggs, sperm and gametes. Even more in particular, the ‘aquatic animal’ means animals of the following species: (i) fish belonging to the superclass Agnatha and to the classes Chondrichthyes, Sarcopterygii and Actinopterygii, (ii) aquatic molluscs belonging to the phylum Mollusca; and (iii) aquatic crustaceans belonging to the subphylum Crustacea. Even more in particular, the aquatic animal according to any aspect of the present invention may be aquatic animals used in aquaculture. Some non-limiting examples of aquatic animals according to any aspect of the present invention include barramundi, carp, catfish, halibut, marbled crayfish, marine and brackish fishes, marine shrimp, mitten crabs, mussels, oysters, pangasius, rainbow trout, salmonids, scallops, sea bass, sea bream, soft-shelled crabs, soft-shelled turtles, tiger prawns, tilapia, turbot, white-leg prawn, and other decapod crustaceans, bivalves and gastropods.

As used herein, the term ‘animal-derived product’ refers to products that originate from animals, particularly aquatic animals. In particular, the term ‘test animal-derived product’ refers to the sample or subject in question that is to undergo the method according to any aspect of the present invention. These products from animals may include meat and meat products, also including eggs, fat, flesh, blood, processed meat and lesser-known products, and non-food products such as fibre (shells, scales and the like). Animal-derived products may also include products that can be made using animal products (e.g. fish oil) such as tablets, powder and such. In one example, the animal-derived product is meat, eggs, blood, brain, shell, scale, skin, tissue, abdominal muscle tissue or any other tissue or sample that provides genomic DNA. In particular, the animal-derived product is meat, skin, blood, trimmings or any organ from the aquatic animal. In particular, trimmings are used as biproducts for fish meal/oil which end up in the animal feed industry or pets. In one example, the animal-derived product sample may be a single type of meat, different types of meat, a single part of a type of meat, different parts of a single type of meat or different parts of different types of meat. The sample may be from any biological entity having a DNA genome and DNA genome methylation. In particular, the methylation site is a CpG site.

The term “test” used in conjunction with the term subject and/or animal in the present disclosure refers to an entity that is subjected to the method according to any aspect of the present invention and is the basis for an analysis application of the present invention. An “(individual) test subject”, an “(individual) group of test subjects” or a “test profile” or an ‘test animal derived product’ is therefore a (individual) subject or group of subjects being tested according to the invention or a profile being obtained or generated in this context. Conversely, the term “reference” or ‘control’ shall denote, mostly predetermined, entities which are used for a comparison with the test entity. For example, the term ‘reference animal’ used interchangeably with ‘control animal’ refers to an animal of the same biological taxon used for comparison or as a control in reference to the ‘test animal’. Similarly, the term ‘sample’ and/or ‘test animal-derived product sample’ used in accordance with any aspect of the present invention refers to an entity that may be subject to the method of the present invention. In particular, a sample may be any (test) animal-derived product that may be subject to the method of the present invention to determine if the test animal has had or has contact with at least one antibiotic and/or veterinary chemical from which the test animal-derived product is obtained from. For example, an animal-derived product sample may be a piece of fish tested according to any aspect of the present invention to determine if the test animal has had contact with at least one antibiotic and/or veterinary chemical from which the test animal-derived product is obtained from. In another example, a (test) animal-derived product sample may be fish eggs which is tested according to any aspect of the present invention to determine if the test animal currently still has contact with at least one antibiotic and/or veterinary chemical from which the test animal-derived product is obtained from. Blockchain may also be used to make the information easily available for the consumer.

As used herein, the term “comprising” is to be construed as encompassing both “including” and “consisting of”, both meanings being specifically intended, and hence individually disclosed aspects of the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles. The DNA segment may also include specific pre-selected methylation sites.

As used herein, the term “pre-selected methylation sites” refers to methylation sites that were selected from genes or regions that showed the highest degree of methylation variation during the training of the method and fulfils certain quality criteria such as a minimum sequencing coverage of ≥5× were considered and for ≥5 qualified CpG sites. Additionally, genes that have an average methylation level <0.1 or an average methylation level >0.9 can be excluded due to their limited dynamic range. “Reference methylation profiles” may be defined on the basis of multiple training samples using multivariate statistical methods, such as such as Principal Component analysis or Multi-Dimensional Scaling.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined. Accordingly, the term “methylation profile” or also “methylation pattern” refers to the relative or absolute concentration of methylated C residues or unmethylated C residues at any particular stretch of residues in the genomic material of a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., the DNA from a sample nucleic acid from a test subject) are methylated as compared to another sequence from a different region or from a different individual (e.g., relative to normal nucleic acid or to the standard nucleic acid of the reference sequence), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to an aquatic animal derived product or aquatic animal that has not had contact with antibiotics and/or veterinary chemicals.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to an aquatic animal derived product or aquatic animal that has not had contact with antibiotics and/or veterinary chemicals.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro. Some of these sites may be hypermethylated and some may be hypomethylated in an animal that was brought into contact with an antibiotic and/or veterinary chemical compared to a cell with no contact with an antibiotic and/or veterinary chemical.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745). In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

As used herein, a “methylated nucleic acid molecule” refers to a nucleic acid molecule that contains one or more nucleotides that is/are methylated.

The term ‘epigenetic change’ as used herein refers to a chemical (e.g., methylation) change or protein (e.g., histones) change that takes place to a gene body or a promoter thereof. Through epigenetic changes, environmental factors like. diet, stress and prenatal nutrition can make an imprint on genes passed from one generation to the next.

As used herein, the term “genomic material” refers to nucleic acid molecules or fragments of the genome of the subject or group of subjects. In particular, such nucleic acid molecules or fragments are DNA or RNA or hybrids thereof, and most preferably are molecules of the DNA genome of a subject or group of subjects.

As used herein, the “DNA sample” refers to the DNA extracted from the terrestrial animal according to any aspect of the present invention using known methods in the art.

In particular, when there is differential methylation detected in a test animal, that is to say that the test animal and/or a product derived from the test animal displays hypermethylation or hypomethylation at, at least one CpG site in comparison to the control (i.e., an aquatic animal without contact with at least one antibiotic and/or veterinary chemical), then the test animal has or has had contact with at least one antibiotic and/or veterinary chemical. The difference in methylation according to any aspect of the present invention may be hypomethylation or hypermethylation.

A “biological sample” in context of the invention may comprise any biological material obtained from the subject or group of subjects that contains genomic material, and may be liquid, solid or both, may be tissue or bone, or a body fluid such as blood, etc. In particular, the biological sample useful for the present invention may comprise biological cells or fragments thereof. More in particular, the biological sample is selected from the group consisting of blood, brain, meat, shell and any other tissue or sample that provides genomic DNA.

The one or more pre-selected methylation sites in (a) are methylation sites associated with tissue specific gene expression, preferably wherein the pre-selected methylation sites are associated with gene expression of one distinct tissue.

The tissue may be selected from
(i) metabolic tissue such as gut or abdominal tissue, said gut tissue preferably being ileum or jejunum,
(ii) muscular tissue,
(iii) skin tissue, and
(iv) organ tissue.

As used herein the term ‘antibiotic’ refers to any medicine that may be fed to the terrestrial animal for therapeutic and/or preventive purposes. The antibiotic may be administered by any method known in the art. The antibiotic may be fed orally to the aquatic animal according to any aspect of the present invention in the animal feed, or water where the aquatic animal is farmed such that it is ingested or used as a bath for external body infections. In another example, the antibiotic may be injected into the aquatic animal. A skilled person would understand the best way to provide the antibiotic to the animal based on the specific biological taxon of the animal, the type of antibiotic and the disease to be treated or prevented. In particular, the antibiotic according to any aspect of the present invention may be selected from the group of classes consisting of amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphonic acid derivatives, pleuromutilins, polymyxins, polypeptides, quinolones, rifamycins, riminofenazines, steroid antibacterials, streptogramins, sulfonamides, tetracyclines, and trimethoprim. More in particular, the antibiotic may be selected from the group consisting of tetracycline and fluoroquinolones, particularly norfloxacin.

The test animal according to any aspect of the present invention may be fed with at least one or more antibiotics mentioned above simultaneously or consecutively. The contact of antibiotics with the aquatic animal may bring about epigenetic changes, at least DNA methylation changes, that may then be determined using the method according to any aspect of the present invention. The concentration of antibiotics in each dose and/or the period of time the antibiotic has been given to the test animal may affect the extend of differential methylation in the test animal relative to the control animal. It is within the knowledge of a skilled person to determine the concentration of each dose and the period of antibiotic exposure that the test animal requires depending on whether the antibiotic is given for preventive or therapeutic measures.

As used herein the term ‘veterinary chemical’ refers to drugs or medicines used to treat or prevent disease, injury and pests in animals in aquaculture. In particular, ‘veterinary chemical’ may refer to an anti-parasitic, an anti-viral, a feed additive, a water additive, a disinfectant, glutaraldehyde, formalin, mixtures thereof and the like. The veterinary chemical may be administered by any method known in the art to the aquatic animal used in aquaculture.

The test animal used in the method according to any aspect of the present invention may be brought into contact with both an antibiotic and a veterinary chemical simultaneously and/or consequently. The change in the internal environment of the test animal leads to an epigenetic change and this can be determined using the method according to any aspect of the present invention.

In particular, in the method according to any aspect of the present invention, in step (a) the methylation status of at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 CpG sites are determined. A skilled person would be capable of determining the number of CpG sites that need to be used in step (a) according to any aspect of the present invention. Even more in particular, the methylation status of at least two CpG sites are determined in step (a) of the method according to any aspect of the present invention.

The method according to any aspect of the present invention, further comprises the step of:
(i) performing bisulfite modification to the DNA sample before step (a).

‘Bisulfite treatment’ of genomic DNA used interchangeably with the term ‘bisulfite modification’, refers to the treatment of the genomic DNA with a deaminating agent such as a bisulfite that may be used to treat all DNA, methylated or not. In particular, the term “bisulfite” as used herein encompasses any suitable type of bisulfite, such as sodium bisulfite, or other chemical agents that are capable of chemically converting a cytosine (C) to an uracil (U) without chemically modifying a methylated cytosine and therefore can be used to differentially modify a DNA sequence based on the methylation status of the DNA, e.g., U.S. Pat. Pub. US 2010/0112595. As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

In context of the present invention also any “non-bisulfite-based method” and “non-bisulfite-based quantitative method” are comprised to test for a methylation status at any given methylation site to be tested. Such terms refer to any method for quantifying methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acid using agents that bind nucleic acid based on methylation status. The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

Accordingly, before step (a) according to any aspect of the present invention is carried out, the genomic DNA contained/obtained or extracted from the cell, is first bisulfite treated.

An alternative method available in the art may be used instead of bisulfite treatment. A skilled person will understand which other methods to use. In one example, TET-assisted pyridine borane sequencing (TAPS) may be used for detection of 5 mC and 5 hmC (Yibin Liu, et al., Nature Biotechnology, 37:424-429 (2019).

According to a further aspect of the present invention, there is provided a method of determining if a test animal and/or a test animal from which a product is derived has been treated and/or is currently undergoing treatment with at least one antibiotic, and if so, determining the distinct class of antibiotics with which the test animal is being treated and/or is currently undergoing treatment, the method comprising:
(a) determining a test methylation profile from the genomic material contained in a biological sample obtained from the test animal and/or the animal-derived product sample; and
(b) comparing the test methylation profile obtained from (a) with one or more predetermined reference methylation profiles, wherein each of the predetermined reference methylation profiles is from a different animal of the same biological taxon as the test animal, and each of the different animals was treated with a different class of antibiotics,
   wherein if the test methylation profile of (a) is significantly similar to one of the predetermined reference methylation profiles, the test animal is confirmed for being treated with the same class of antibiotics with which the animal with the predetermined reference profile is treated with and the test animal has been confirmed for having been treated and/or is currently undergoing treatment with the distinct class of antibiotics; and
   wherein the test animal is an aquatic animal.

The distinct classes of antibiotics are amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphonic acid derivatives, pleuromutilins, polymyxins, polypeptides, quinolones, rifamycins, riminofenazines, steroid antibacterials, streptogramins, sulfonamides, tetracyclines, and trimethoprim.

In one example, a panel of pre-determined reference profiles may be prepared for different animals to be used as a control where each animal has been contacted with a different class of antibiotics and/or each part of the animal (i.e. tissue, muscle, blood, skin) has its own unique pre-determined methylation reference profile that also forms a part of the panel of pre-determined reference profiles. Different animals from the same biological taxon as the test animal, each being treated with a different class of antibiotic may have its own panel of pre-determined reference profiles for each part of the animal or animal-derived product that is used as the genomic material. For example, each panel may be specific for a single animal in contact with a first antibiotic and/or veterinary chemical where each reference profile may be distinct for a part of the animal from which the genomic material is extracted. There will thus be a compilation of panels of pre-determined reference profiles, each panel specific for one control animal of the same biological taxon as the test animal, the control animal being in contact with or is in contact with a first, second, third and the like antibiotic and/or veterinary chemical. When a test methylation profile from an unknown animal-derived product sample is obtained, this is then compared with the different panels of pre-determined reference profiles for the same animal taxon as the test animal to determine the distinct class of antibiotic and/or veterinary chemical the test animal is or was in contact with.

According to yet another aspect of the present invention there is provided a method of determining if a test animal and/or a test animal from which a product is derived has been treated and/or is currently undergoing treatment with at least one antibiotic, and if so, determining if the antibiotic is used as a growth promotant or as a therapeutant, the method comprising:
(a) determining a test methylation profile from the genomic material contained in a biological sample obtained from the test animal and/or the animal-derived product; and
(b) comparing the test methylation profile obtained from (a) with one or more predetermined reference methylation profiles, wherein each of the predetermined reference methylation profiles is from a different control animal of the same biological taxon as the test animal, and each of the different control animals was treated with the antibiotic as a growth promotant or therapeutant,
   wherein if the test methylation profile of (a) is significantly similar to one of the predetermined reference methylation profiles, the test animal is confirmed for being treated with the antibiotic in the same way that the control animal with the similar predetermined reference profile is treated and the test animal has been confirmed for having been treated and/or is currently undergoing treatment with the antibiotic as a growth promotant or therapeutant; and wherein the test animal is an aquatic animal.

As used herein the term ‘growth promotant’ refers to the antibiotic being used to help increase the efficiency of animal production by increasing weight gain and product output. The antibiotic may be used as a growth promotant in contrast to it being used as a therapeutant (i.e. for treatment of a disease)

According to a further aspect of the present invention, there is provided a method of determining if a test animal from which a product is derived underwent a withdrawal period of no treatment with at least one antibiotic and/or veterinary chemical prior to the product being obtained, the method comprising:
(a) determining a test methylation profile from the genomic material contained in a biological sample obtained from the animal-derived product; and
(b) comparing the test methylation profile obtained from (a) with at least two predetermined reference methylation profiles, wherein at least one of the predetermined reference methylation profiles is from a control animal that underwent a withdrawal period and at the other one of the predetermined reference methylation profiles is from a control animal of the same biological taxon as the test animal, that did not undergo a withdrawal period before the product was obtained,
   wherein if the test methylation profile of (a) is significantly similar to the predetermined reference methylation profile, the test animal is confirmed for having undergone a withdrawal period or not; and
   wherein the test animal is an aquatic animal.

As used herein, the term ‘withdrawal period’ refers to the period from the time point where the animal is no longer fed the antibiotic and/or veterinary chemical to the point where the remaining antibiotic is broken down in the body until it becomes a non-functional agent and is finally, eliminated from the body of the animal. Withdrawal periods of different antibiotics may vary from 1 or 2 days to couple of weeks. A “withdrawal” period is required from the time antibiotics are administered until it is legal to slaughter or kill the animal or to derive products from the animal. The time it therefore takes the body to break down the antibiotic until it is no longer functional, or present is called the withdrawal time (or withdrawal period). Once the withdrawal period has passed the antibiotic has been eliminated from the animal's system.

According to another aspect of the present invention, there is provided a use of DNA methylation profiling for determining if a test animal and/or a test animal from which a product is derived has been treated and/or is currently undergoing treatment with at least one antibiotic and/or veterinary chemical, wherein the test animal is an aquatic animal and the veterinary chemical is an anti-parasitic, an anti-viral, a feed additive, a water additive, a disinfectant, glutaraldehyde, and/or formalin used in aquaculture.

### BRIEF DESCRIPTION OF FIGURES

FIG. 1 shows the Principal component analysis (PCA) of multiple samples of tetracycline treatment. Based on antibiotic-specific methylations in 1754, different DNA segments. Red: Control; Green: TetraA (tetracycline at 10 mg/kg for 7 days); Blue: TetraB (tetracycline at 5 mg/kg for 6 weeks).

FIGS. 2a and 2b are heatmaps of the methylation ratios of the CpGs of two genes. The methylation rate is given on a scale from 0 (blue) to 1 (red). Blue shows only unmethylated cytosine, while red represents exclusively methylated cytosine. Each row shows the methylation status of a sample, each column shows a CpG. The groups Control, TetraA (tetracycline treatment at 10 mg/kg for 7 days) and TetraB (tetracycline treatment at 5 mg/kg for 6 weeks) are compared. (a) Shows a genomic region with five CpGs. (b) Represents a genomic region with 9 CpGs.

FIG. 3 shows the Principal component analysis (PCA) of multiple samples of norfloxacin treatment. Based on antibiotic-specific methylation patterns in 937 various DNA segments. Red: Control; Green: NorA (Norfloxacin at 10 mg/kg for 7 days); Blue: NorB (Norfloxacin at 5 mg/kg for 4 weeks).

FIGS. 4a and b are heatmaps of the methylation ratios of the CpGs of two genes. The methylation rate is given on a scale from 0 (blue) to 1 (red). (blue) shows only unmethylated cytosines; (red) represents exclusively methylated cytosine. Each row shows the methylation status of a sample, each column shows a CpG. Comparison of the control with NorA (Norfloxacin for 7 days at 10 mg/kg) and NorB (Norfloxacin for 4 weeks at 5 mg/kg). (a) Showed in gene with eight CpGs. (b) Represents a gene that also has eight CpGs.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Marble crayfish collected from a population at Lake Reilinger, Germany were collected for the experiment and reared from juveniles in the laboratory. A total of 30 adult marbled crayfish were reared in one of 5 treatments with replicates as outlined in Table 1:
1) Control—no antibiotic treatment
2) TetraA—Acute tetracycline administration at 10 mg/kg of feed for 7 days
3) TetraB—Prophylactic tetracycline administration at 5 mg/kg of feed for 42 days
4) NorA—administered at 10 mg/kg of feed for 7 days
5) NorB—administered at 5 mg/kg of feed for 28 days

All other rearing variables were kept consistent for all treatment groups. Crayfish were reared in plastic containers with 20° C. tap water, which was replaced every two weeks, exposed to natural day-night light rhythms and daily feeding.

A subset of abdominal tissue genomic DNA samples (Table 1) was utilized for analysis of the Agilent Sure Select Methyl-Seq Assay. Abdominal tissue was extracted from marble crayfish directly after treatment duration, DNA was then extracted as previously described (Tönges et al, 2021).

TABLE 1
Number of replicate adult marble crayfish from
each treatment utilized for methylation analysis
of genomic DNA from abdominal tissue samples.

| | Control | TetraA | TetraB | NorA | NorB |
|---|---|---|---|---|---|
| Adult | 3 | 3 | 4 | 3 | 3 |

Library preparation was carried out as described in the Agilent's Sure Select Methyl-Seq Target Enrichment System for Illumina Multiplexed Sequencing Protocol (version E0, April 2018) to specifically analyze 361 genes. These genes were previously characterized as variably methylated genes in the marble crayfish genome, showing differential site-specific methylation patterns in marble crayfish from different wild populations (Gatzmann et al., 2018; Tönges et al., 2021b). This indicates that the variable methylations of these genes could be environmentally influenced and may therefore also be influenced by treatments such as antibiotic exposure.

Quality controls were performed, and sample concentrations were measured on a 2,200 TapeStation (Agilent). Multiplexed samples were submitted to the DKFZ High Throughput Sequencing core facility and sequenced on a HiSeq system (Illumina). For data analysis, the 361 sequenced genes were mapped to the bisulfite sequencing datasets of the entire marble crab genome (Gatzmann et al., 2018; Xi and Li 2009).

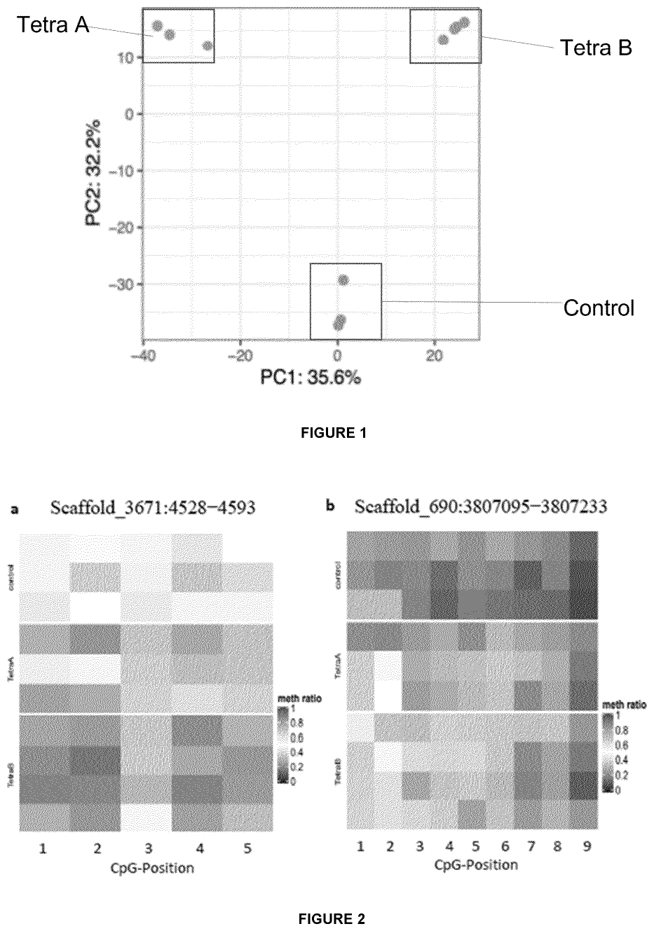

To identify antibiotic-specific methylation ratios in genomic DNA from abdominal tissue, the average methylation of each sample within 1 kb of DNA segments was calculated. A one-factorial ANOVA was then used to investigate whether the average methylation rates differ significantly between the two treatment groups with tetracycline compared to the control. The analysis comparing tetracycline treatments to the control yielded 1754 DNA segments that were significantly differentially methylated (p<0.05) (FIG. 1). The PCA shows a robust separation between the treated and untreated samples and between the samples of the two different groups treated with tetracycline. The main component 1 (35.6%) separates the two treatment groups, TetraA and TetraB. In addition, the main component 2 (32.2%) clearly separates the samples treated with tetracycline from the control (FIG. 1).

The data sets of the 1754 DNA segments were further analyzed and only the CpG positions covered in all samples with at least 10 reads were used. In addition, the CpGs that were either completely methylated or unmethylated (average methylation <0.2 or >0.8) were filtered out. Subsequently, a Kruskal Wallis test revealed 432 DNA segments with significant differences between the control and the treated animals (p<0.05).

Within the five CpGs of the first gene (Scaffold_3671: 4528-4593), the samples of the control appear to have higher overall methylation rates, while the Tetra B group shows the lowest methylation rates on average (FIG. 2a). The nine CpGs of the second gene (Scaffold_690: 3807095-3807233) have, on average, lower methylation in the samples of the control. Methylations within the TetraA group appear to be increased in comparison and the highest methylation rates are seen in the TetraB group. There is no directed methylation, instead the methylation differences are strongly localized (FIG. 2b).

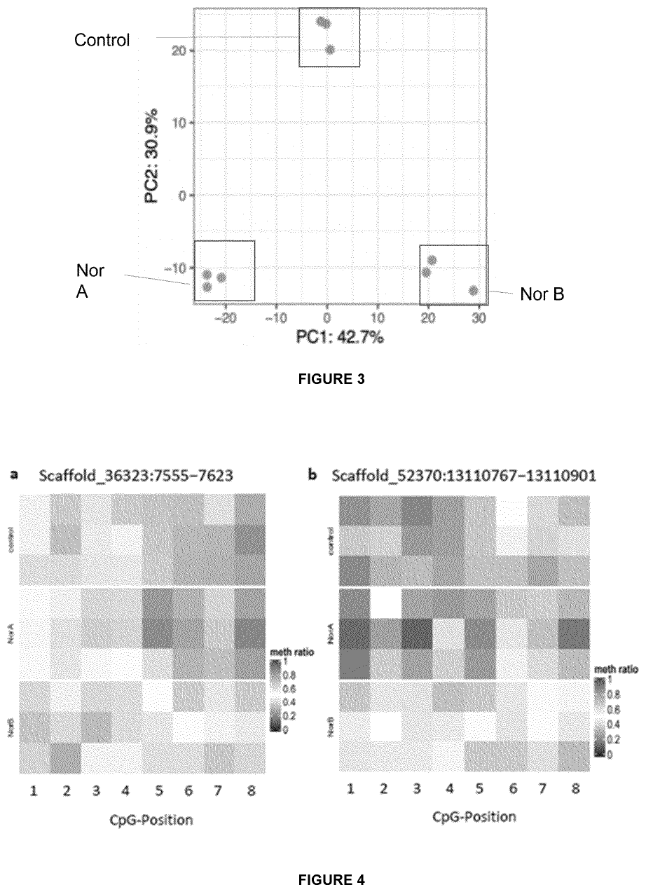

The same approach was used to analyze antibiotic-specific methylation patterns of the different norfloxacin samples. The analysis identified 937 different DNA segments that are significantly differentially methylated (p<0.05) (FIG. 3). The PCA showed a robust separation between the two different norfloxacin groups as well as between the control and the norfloxacin treated samples. The main component 1 (42.7%) separates the two treatment groups, NorA and NorB, from each other.

Further pronounced separation is caused by main component 2 (30.9%), which shows the difference between the control and the samples treated with norfloxacin (FIG. 3).

The DNA segments of the PCA were further analyzed and only the CpG positions that have at least 10 reads in all samples were reused. Furthermore, the CpGs that are completely unmethylated or methylated (average methylation <0.2 or >0.8) were filtered out. As a result, a Kruskal Wallis test revealed 71 DNA segments with significant differences between the control and the treated animals (p<0.05).

The average methylations indicate individual DNA segments with moderate methylation differences between the control and the two treatment groups. The effect sizes were determined using Cohen's d, with 4 DNA segments showing a small effect (d>0.2) and all others a very small effect (d<0.02). Analysis of DMRs reveals no significant differences between treatment groups and control (p>0.05). Nevertheless, treatment-specific differences in the DMRs have been demonstrated. These methylation differences are exemplified for the CpGs of two genes (Scaffold_36323: 7555-7623, Scaffold_52370: 13110767-13110901) (FIG. 4).

In the first gene (Scaffold_36323: 7555-7623), the CpGs of the control and the Nor A group appear to have average similar methylation ratios. On the other hand, when comparing the control and NorA group with the NorB group, the latter shows higher methylation rates in all CpGs. These differences are particularly evident in CpGs one to four and seven (FIG. 4a). The average methylation rates of the second gene (Scaffold_52370: 13110767-13110901) show no significant difference between the control and the NorA group. Group NorB, on the other hand, shows higher methylation ratios of all CpGs, also in this gene (FIG. 4 b). The largest differences in methylation can be seen when comparing the control with the samples from the four-week treatment with 5 mg/kg norfloxacin (NorB). In both genes, the average methylation was increased by the treatment. In the seven-day treatment with 10 mg/kg norfloxacin (NorA), on the other hand, no significant difference to the control can be observed. Overall, highly localized methylation patterns can be observed (FIG. 4).

In summary, DNA methylation analysis was able to separate both tetracycline and norfloxacin treated marble crayfish from controls.

1. A method of determining if a test animal and/or a test animal from which a product is derived has been treated and/or is currently undergoing treatment with at least one antibiotic and/or veterinary chemical, the method comprising:
(a) determining a test methylation profile from the genomic material contained in a biological sample obtained from the test animal and/or the animal-derived product; and
(b) comparing the test methylation profile obtained from (a) with a reference methylation profile obtained from a control animal of the same biological taxon of the test animal, where the control animal was not treated and/or is not currently undergoing treatment with at least one antibiotic and/or veterinary chemical,
wherein a difference in the test methylation profile of (a) compared to the reference methylation profile from the control animal, is indicative of the test animal having been treated and/or is currently undergoing treatment with at least one antibiotic and/or veterinary chemical; and
wherein the test animal is an aquatic animal and
the veterinary chemical is an anti-parasitic, an anti-viral, a feed additive, a water additive, a disinfectant, glutaraldehyde, and/or formalin used in aquaculture.

2. The method according to claim 1, wherein the difference is hypomethylation or hypermethylation.

3. A method of determining if a test animal and/or a test animal from which a product is derived has been treated and/or is currently undergoing treatment with at least one antibiotic and/or veterinary chemical, the method comprising:
(a) determining the methylation status of at least one CpG site in genomic material contained in a biological sample obtained from the test animal,
(b) comparing the methylation status of the CpG site from (a) with that of a control animal of the same biological taxon as the test animal, which was not treated and/or is not currently undergoing treatment with at least one antibiotic and/or veterinary chemical,
wherein a difference in the test methylation status of (a) in the test animal compared to the CpG site in the control animal is indicative of the test animal having been treated and/or is currently undergoing treatment with at least one antibiotic and/or veterinary chemical; and
wherein the test animal is an aquatic animal; and
the veterinary chemical is an anti-parasitic, an anti-viral, a feed additive, a water additive, a disinfectant, glutaraldehyde, and/or formalin used in aquaculture.

4. The method according to claim 3, wherein the difference in methylation status is hypomethylation or hypermethylation of the CpG site in the test animal and the hypomethylation or hypermethylation of the CpG site is indicative of the test animal having been treated and/or is currently undergoing treatment with at least one antibiotic and/or veterinary chemical.

5. The method according to claim 1, wherein the animal-derived product is meat, skin, blood, trimmings or at least one organ from the aquatic animal.

6. The method according to claim 1, wherein the aquatic animal selected from the group consisting of fish, crustacean, and molluscs.

7. The method according to claim 1, wherein the aquatic animal is selected from the group consisting of carps, salmonids, tilapias, catfish, marine and brackish fishes, soft-shelled turtles, barramundi, marine shrimp, mitten crabs, marbled crayfish and other decapod crustaceans, bivalves and gastropods.

8. The method according to claim 1, wherein the biological sample is selected from the group consisting of skin, scale, tissue, abdominal muscle tissue or any sample that provides genomic DNA.

9. The method according to claim 1, wherein the antibiotic is selected from the group of classes consisting of amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphonic acid derivatives, pleuromutilins, polymyxins, polypeptides, quinolones, rifamycins, riminofenazines, steroid antibacterials, streptogramins, sulfonamides, tetracyclines, and trimethoprim.

10. A method of determining if a test animal and/or a test animal from which a product is derived has been treated and/or is currently undergoing treatment with at least one antibiotic, and if so, determining the distinct class of antibiotics with which the test animal is being treated and/or is currently undergoing treatment, the method comprising:
(a) determining a test methylation profile from the genomic material contained in a biological sample obtained from the test animal and/or the animal-derived product sample; and
(b) comparing the test methylation profile obtained from (a) with one or more predetermined reference methylation profiles, wherein each of the predetermined reference methylation profiles is from a different animal of the same biological taxon as the test animal, and each of the different animals was treated with a different class of antibiotics,
wherein if the test methylation profile of (a) is significantly similar to one of the predetermined reference methylation profiles, the test animal is confirmed for being treated with the same class of antibiotics with which the animal with the predetermined reference profile is treated with and the test animal has been confirmed for having been treated and/or is currently undergoing treatment with the distinct class of antibiotics; and
wherein the test animal is an aquatic animal.

11. The method according to claim 10, wherein the distinct classes of antibiotics are amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphonic acid derivatives, pleuromutilins, polymyxins, polypeptides, quinolones, rifamycins, riminofenazines, steroid antibacterials, streptogramins, sulfonamides, tetracyclines, and trimethoprim.

12. A method of determining if a test animal and/or a test animal from which a product is derived has been treated and/or is currently undergoing treatment with at least one antibiotic, and if so, determining if the antibiotic is used as a growth promotant or as a therapeutant, the method comprising:
(a) determining a test methylation profile from the genomic material contained in a biological sample obtained from the test animal and/or the animal-derived product; and
(b) comparing the test methylation profile obtained from (a) with one or more predetermined reference methylation profiles, wherein each of the predetermined reference methylation profiles is from a different control animal of the same biological taxon as the test animal, and each of the different control animals was treated with the antibiotic as a growth promotant or therapeutant,
wherein if the test methylation profile of (a) is significantly similar to one of the predetermined reference methylation profiles, the test animal is confirmed for being treated with the antibiotic in the same way that the control animal with the similar predetermined reference profile is treated and the test animal has been confirmed for having been treated and/or is currently undergoing treatment with the antibiotic as a growth promotant or therapeutant; and
wherein the test animal is an aquatic animal.

13. A method of determining if a test animal from which a product is derived underwent a withdrawal period of no treatment with at least one antibiotic and/or veterinary chemical prior to the product being obtained, the method comprising:
(a) determining a test methylation profile from the genomic material contained in a biological sample obtained from the animal-derived product; and
(b) comparing the test methylation profile obtained from (a) with at least two predetermined reference methylation profiles, wherein at least one of the predetermined reference methylation profiles is from a control animal that underwent a withdrawal period and at the other one of the predetermined reference methylation profiles is from a control animal of the same biological taxon as the test animal, that did not undergo a withdrawal period before the product was obtained,
wherein if the test methylation profile of (a) is significantly similar to the predetermined reference methylation profile, the test animal is confirmed for having undergone a withdrawal period or not; and
wherein the test animal is an aquatic animal; and
the veterinary chemical is an anti-parasitic, an anti-viral, a feed additive, a water additive, a disinfectant, glutaraldehyde, and/or formalin used in aquaculture.

14. A method of using DNA methylation profiling, comprising determining with DNA methylation profiling if a test animal and/or a test animal from which a product is derived has been treated and/or is currently undergoing treatment with at least one antibiotic and/or veterinary chemical, wherein the test animal is an aquatic animal and the veterinary chemical is an anti-parasitic, an anti-viral, a feed additive, a water additive, a disinfectant, glutaraldehyde, and/or formalin used in aquaculture.
